(19) United States
(12) Patent Application Publication
(10) Pub. No.: US 2008/0119907 A1
(43) Pub. Date: May 22, 2008

(54) RENAL FUNCTION MODULATION VIA APPLICATION OF ELECTRICAL ENERGY STIMULATION

(75) Inventor: Jeffrey E. Stahmann, Ramsey, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc., St. Paul, MN (US)

(21) Appl. No.: 11/562,436

(22) Filed: Nov. 22, 2006

(51) Int. Cl. A61N 1/05

(52) U.S. Cl. 607/40

(57) ABSTRACT

Renal function modulation via application of electrical energy stimulation is discussed. The electrical energy stimulation includes a frequency equal to or greater than about 1 KHz and is injected between a first electrode and a second electrode, at least one of which is internally disposed proximal to a subject's kidney such that a substantially large portion of the stimulation passes through at least one of a glomerulus, a Bowman's capsule, a macula densa, a tubule, a peritubular capillary network, a collecting duct, an afferent arteriole, an efferent arteriole, or a renal granular cell. The electrical energy stimulation modulates one or more renal functions. One or more parameters associated with the one or more renal functions are measured and used to, among other things, determine a kidney status indicative signal or control the electrical energy stimulation applied.

### TECHNICAL FIELD

This patent document pertains generally to medical systems and methods. More specifically, this patent document pertains to renal function modulation via application of electrical energy stimulation.

### BACKGROUND

Kidneys are vital organs that perform many functions including regulation of water and electrolytes, excretion of metabolic wastes and bioactive substances, and regulation of arterial blood pressure, red blood cell production and vitamin D. Every day, the kidneys process about 200 quarts of blood to sift out about 2 quarts of waste products and water. The waste and extra water become urine, which flows to one's bladder through tubes called urethers. The bladder stores the urine until it is excreted. The wastes in the blood come from the normal breakdown of active bodily tissues and from consumed food. The body uses the food for energy and self-repairs. After the body has taken what it needs from the food, waste is sent to the blood. If the kidneys do not remove this waste, the waste builds-up in the blood and may damage the body.

The actual filtering in the kidneys occurs via tiny units therein called nephrons. In each nephron, a group of interconnected capillary loops, called the glomerulus, filters the blood and produces a fluid, called the filtrate. The filtrate is similar to blood plasma but contains very little total protein. Unlike large proteins (e.g. albumin), inorganic ions and low-molecular-weight organic solutes are freely filtered by the glomerulus into the filtrate. Since the inorganic ions and low-molecular-weight organic solutes are freely filtered, their concentrations in the filtrate are very similar to their concentration in blood plasma.

The filtrate leaving the glomerulus contains a combination of waste materials that need to be removed from the body, other solutes (e.g. electrolytes)—some of which need to be removed from the body and some of which need to be retained by the body, and water—most of which needs to be retained by the body. To affect the removal and retention these substances, the filtrate leaving the glomerulus empties into a tiny tube called a tubule. Several processes occur within the tubule. These processes combined with filtration by the glomerulus affect proper removal and retention of the various solutes and water. Most of the water and other solutes (e.g. glucose, electrolytes, bicarbonate) are reabsorbed as the filtrate moves though the tubule. The process of reabsorption is critical since without it, the body would quickly dehydrate and suffer electrolyte and pH imbalances. Secretion occurs within the tubule and is critical for many processes, for example, pH balance (hydrogen ion secretion) and potassium balance. Some of the water and solutes (e.g. urea) pass through the tubule, thus producing urine.

In addition to the secreted substances described above, the kidneys release important hormones, such as erythropoietin (EPO), which stimulates bone marrow to make red blood cells; renin, which regulates blood pressure; and calcitriol, which helps maintain calcium for bones and for normal chemical balance in the body. Still other functions performed by the kidneys include maintenance of the body's control of several important endocrine functions.

Unfortunately, a number of people experience progressively worsening renal failure as a result of a variety of disorders. As one or more of the disorders worsen, a person typically cannot live long without some form of renal (i.e., kidney) therapy. In many instances, the treatment of renal failure attempts to address secondary symptoms of the failure, rather than directly impact the function of the kidneys themselves. For example, diuretics are often given to reduce blood volume and pain medication is often given to alleviate subject discomfort.

End stage renal failure is typically treated by hemodialysis (where the blood is artificially “cleaned” by exchange with a dialysis fluid across a selectively permeable membrane) or by transplantation, both of which have numerous associated drawbacks. Dialysis subjects, for example, must adhere to rigid dialysis schedules that are typically on the order of four hours at a time, three times per week. Dialysis subjects must also restrict fluid intake, follow strictly controlled diets, take daily medications, and endure such things as anemia, abnormal bone metabolism, chronic uremia, and diminished sexual function. An alternative to hemodialysis is transplantation. However, transplantation also has associated drawbacks, including being an inherently risky procedure and the risk of organ rejection. Additionally, transplantation is at the mercy of organ supply, which currently is experiencing growing shortages.

Given the wide range of important functions that the kidneys provide, it is desirable to maintain the kidneys in a state of relative well-being, including modulating kidney function prior to, during, or following renal disease or other degenerative disorders.

### SUMMARY

One embodiment of the present subject matter includes a method for applying a stimulus to at least one of a glomerulus, a Bowman's capsule, a macula densa, a tubule, a peritubular capillary network, a collecting duct, an afferent arteriole, an efferent arteriole, or a renal granular cell within a kidney of a subject. The method includes, among other things, injecting a first electrical energy signal having a frequency between about 1 KHz and about 1 MHz. The first electrode and the second electrode are positioned and configured to direct a substantially large portion of the first electrical signal through at least one of the glomerulus, the Bowman's capsule, the macula densa, the tubule, the peritubular capillary network, the collecting duct, the afferent arteriole, the efferent arteriole, or the renal granular cell, thereby modulating one or more renal functions. In varying embodiments, at least one of the first electrode or the second electrode is disposed within the subject and proximal to the kidney.

One embodiment of the present subject matter includes a system for applying a stimulus to at least one of a glomerulus, a Bowman's capsule, a macula densa, a tubule, a peritubular capillary network, a collecting duct, an afferent arteriole, an efferent arteriole, or a renal granular cell within a kidney of a subject. The system includes, among other things, a first electrode, a second electrode, and an electrical energy delivery circuit. The electrical energy delivery circuit is coupled to the first electrode and the second electrode to deliver a generated first electrical energy signal having a frequency between about 1 KHz and about 1 MHz. The first electrode and the second electrode are positioned and configured to direct a substantially large portion of the first electrical signal through at least one of the glomerulus, the Bowman's capsule, the macula densa, the tubule, the peritubular capillary network, the collecting duct, the afferent arteriole, the efferent arteriole, or the renal granular cell to modulate one or more renal functions.

Advantageously, the present subject matter may keep kidney subjects in a state of relative well-being by preventing, delaying, or minimizing renal conditions including, for example, chronic kidney disease and end stage renal failure via application of internal electrical energy stimulation. The electrical energy stimulation may be used conjunctively or in lieu of drug or other therapies to modulate one or more renal functions. In this way, the electrical energy stimulation provides an option for subjects that respond inadequately to drug therapy, are intolerant of drug therapy, have preference for treatment via electrical energy stimulation, or are non-compliant with drug therapy and may further modulate renal functions that are beyond the reach of existing drug therapy. Yet another advantage of the present subject matter is that it may be configured such that subject action or compliance is not needed for resulting improvement of subject health.

This Summary is an overview of some of the teachings of the present patent document and not intended to be exclusive or exhaustive treatment of the present subject matter. Further details about the present subject matter are found in the detailed description and appended claims. Other aspects and advantages will be apparent to persons skilled in the art upon reading and understanding the following detailed description and viewing the drawings that form a part thereof, each of which are not to be taken in a limiting sense. The scope of the present subject matter is defined by the appended claims and their legal equivalents.

### BRIEF DESCRIPTION OF THE DRAWINGS

In the drawings, like numerals describe substantially similar components throughout the several views. The drawings illustrate generally, by way of example, various embodiments discussed in the present document.

FIG. 1 is a schematic view of a system for delivering electrical energy stimulation to one or more portions of a subject's body, including a subject's kidney(s), according to one embodiment of the present subject matter.

FIG. 2 is a block diagram of a system for delivering electrical energy stimulation to one or more portions of a subject's body, including as a subject's left kidney, according to one embodiment of the present subject matter.

FIG. 3A is a schematic view of a system in the course of delivering electrical energy stimulation in the form of an electric current or an electrical field to a portion of a subject's left kidney, according to one embodiment of the present subject matter.

FIG. 3B diagrammatically illustrates a nephron of a kidney to which electrical energy stimulation may be delivered, according to one embodiment of the present subject matter.

FIG. 4A is a schematic view of kidney structures associated with one or more renal functions that may be modulated via application of electrical energy stimulation, according to one embodiment of the present subject matter.

FIG. 4B is an enlarged view of one or more kidney structures alterable via application of electrical energy stimulation, according to one embodiment of the present subject matter.

FIG. 4C is an enlarged view of various kidney structure transport mechanisms alterable via application of electrical energy stimulation, according to one embodiment of the present subject matter.

FIG. 5 illustrates a method of modulating one or more renal functions using electrical energy stimulation, according to one embodiment of the present subject matter.

### DETAILED DESCRIPTION

The following detailed description of the present subject matter refers to subject matter in the accompanying drawings which show, by way of illustration, specific embodiments in which the present subject matter may be practiced. References to “an”, “one”, or “various” embodiments in this patent document are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

Various embodiments of the present subject matter are provided herein for renal function modulation via application of electrical energy stimulation. The electrical energy stimulation may be used to supplement or in lieu of existing treatments affecting renal function (e.g., drug therapy, hemodialysis or transplantation, among others) to keep kidney subjects in a state of relative well-being by preventing, delaying, or minimizing renal conditions including, for example, chronic kidney disease and end stage renal failure. It is believed that by selectively manipulating (via application of electrical energy stimulation) one or more kidney structures (e.g., a glomerulus, a Bowman's capsule, a macula densa, a tubule, a peritubular capillary network, a collecting duct, an afferent arteriole, an efferent arteriole, or a renal granular cell) that one or more renal functions performed by such structures may be modulated allowing a desired biological response of one or more renal function-associated parameters (e.g., an electrolyte level, a water level, a metabolic waste level (including a creatinine level, a blood urea nitrogen level, or a uric acid level), a pharmacological agent level, a hormone level, a blood pressure level, an erythropoietin level, a vitamin D level, a glucose level, a pH level, or a glomerulus filtration rate level) to be effectuated. By altering the one or more renal function-associated parameters as desired, it is further believed that associated diseases (e.g., hypertension, edema, heart failure, blood electrolyte imbalances, and others) may be treated or prevented.

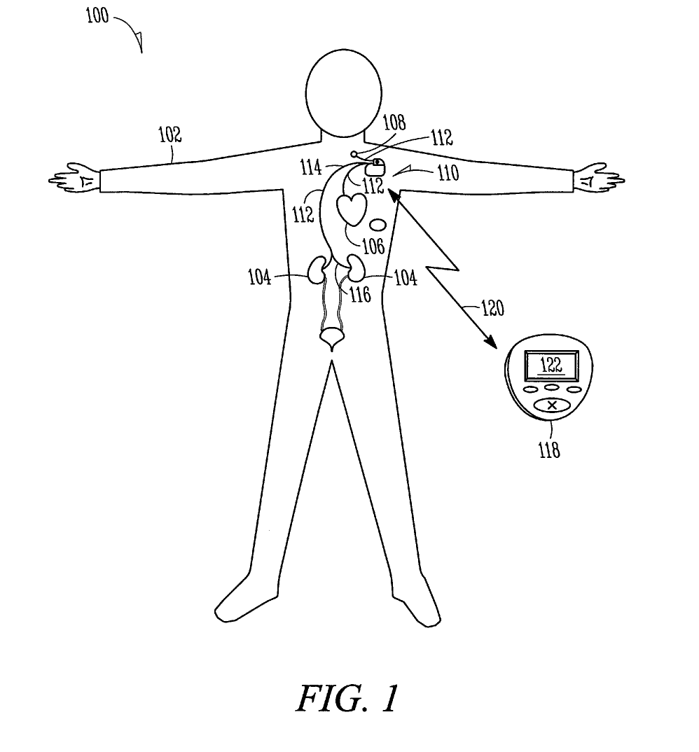

FIG. 1 schematically illustrates one embodiment of a system 100 for delivering electrical energy stimulation to one or more portions of a subject's body 102, such as one or both kidneys 104, the heart 106, or an efferent parasympathetic nerve 108. While not shown, the system 100 may also be configured to deliver electrical energy stimulation to other portions of the subject's body 102, such as the brain or pulmonary regions. In this embodiment, the system 100 includes an implantable medical device (IMD) 110, such as a pulse generator including cardiac therapy capabilities (e.g., capable of providing one or more of bradycardia therapy, tachycardia therapy, or cardiac resynchronization therapy), which is coupled by one or more leads 112 to the kidneys 104, the heart 106, and the efferent para-sympathetic nerve 108. The IMD 110 may be implanted subcutaneously in the subject's chest, abdomen, or elsewhere. Each of the one or more leads 112 extends from a lead proximal end portion 114 to a lead distal end portion 116, the latter of which includes one or more electrodes for delivering the electrical energy stimulation generated by the IMD 110 to the kidney(s) 104, the heart 106, or the efferent parasympathetic nerve 108.

The exemplary system 100 shown also includes an external user-interface 118. The external user-interface 118 may be used to receive information from, or send information to, the IMD 110. For instance, new values for one or more electrical energy parameters (e.g., an energy injection location, an energy injection duration, an energy injection intensity, an energy injection frequency, an energy injection polarity, an energy injection electrode configuration, or an energy injection waveform) applied to one or more kidney structures (e.g., a glomerulus, a Bowman's capsule, a macula densa, a tubule, a peritubular capillary network, a collecting duct, an afferent arteriole, an efferent arteriole, or a renal granular cell) may be manually input into the external user-interface 118 and sent to the IMD 110 so-as-to change a parameter of the electrical energy stimulation resulting in a desired biological response of one or more renal function-associated parameters (e.g., an electrolyte level, a water level, a metabolic waste level (including a creatinine level, a blood urea nitrogen level, or a uric acid level), a pharmacological agent level, a hormone level, a blood pressure level, an erythropoietin level, a vitamin D level, a glucose level, a pH level, or a glomerulus filtration rate level). Additionally, the external user-interface 118 may be used to receive one or more inputs of the subject's 102 health-related information. In certain embodiments, the external user-interface 118 is used to externally process information for the system 100. Using telemetry or other known communication techniques, the external user-interface 118 may wirelessly communicate 120 with the IMD 110. As shown, the external user-interface 118 may include a visual or other display unit 122, such as an LCD or LED display, for textually or graphically relaying information to the subject 102 or a caregiver regarding operation or findings of the system 100.

While the present system 100 may find utility in sensing and/or stimulating many portions of a subject's 102 body, particular attention will hereinafter be made to the present system's 100 use with one or more portions of a subject's kidney(s), and more specifically, with one or more of the glomerulus, the Bowman's the macula densa, the tubule, the peritubular capillary network, the collecting duct, the afferent arteriole, the efferent arteriole, or the renal granular cell.

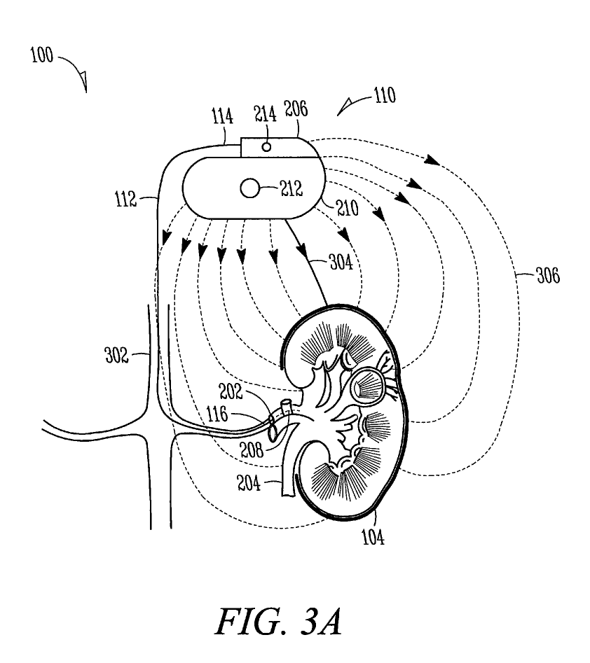

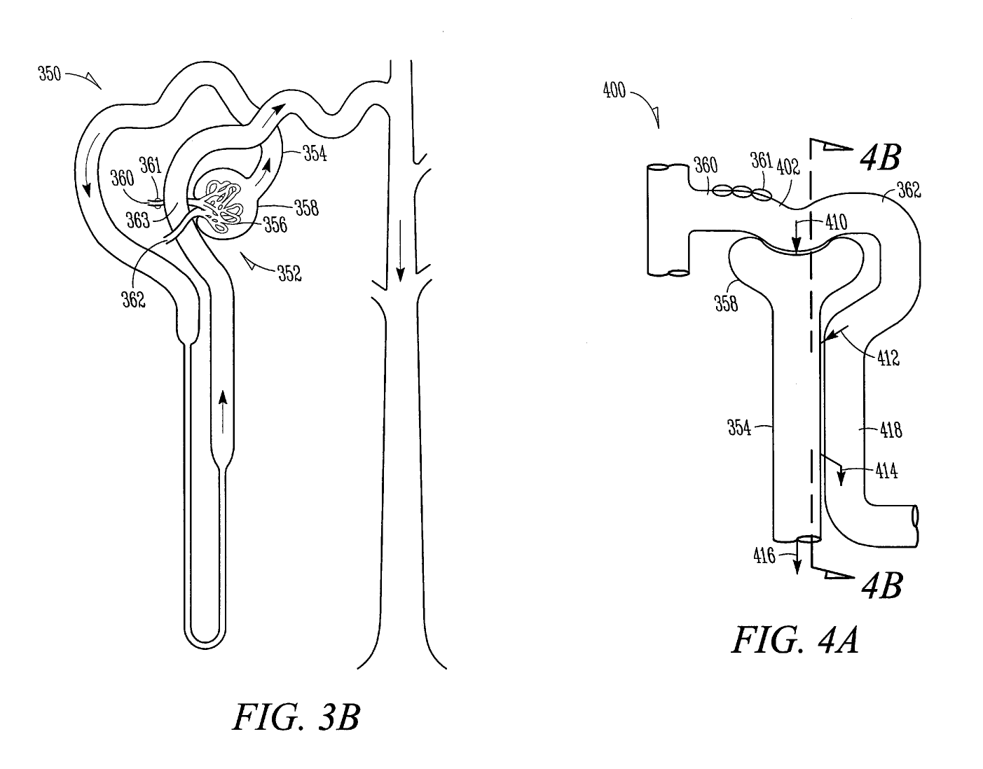

As discussed above, the actual filtering in the kidneys 104 occurs via tiny units therein called nephrons 350 (FIG. 3B). Each kidney has about a million nephrons 350. It is known that the major cause of renal failure is not a change in the filtration properties of working nephrons but rather a decrease in the number of functioning nephrons 350. As some nephrons 350 become diseased, others compensate by enlarging and assuming a portion of the lost function. Over time, more and more of the nephrons 350 become diseased to the point where the working nephrons 350 are unable to provide, among other things, the needed filtration, electrolyte balance, or hormonal balance to the kidney 104 for adequate performance thereof. Such inadequate kidney 104 performance is likely to result in disease-indicative levels of one or more renal function-associated parameters (e.g., an electrolyte level, a water level, a metabolic waste level (including a creatinine level, a blood urea nitrogen level, or a uric acid level), a pharmacological agent level, a hormone level, a blood pressure level, an erythropoietin level, a vitamin D level, a glucose level, a pH level, or a glomerulus filtration rate level).

To restore kidney performance, the present subject matter is provided. It is believed that by artificially stimulating (via the application of electrical energy stimulation) those nephrons 350 and/or associated renal structures, that for various reasons have stopped contributing, or contribute in a reduced fashion, to the overall functions of the kidney 104, renal performance may be affected in a positive way. Additionally, it is believed that electrical energy stimulation of nephrons 350 and/or associated renal structures will provoke normally functioning nephrons 350 and/or associated renal structures into a state of hyperfunctionality thus compensating for renal function lost due to malfunctioning nephrons 350 and/or malfunctioning renal functions associated with such nephrons 350. In various embodiments, the electrical energy stimulation is applied to one or more renal structures (e.g., a glomerulus, a Bowman's capsule, a macula densa, a tubule, a peritubular capillary network, a collecting duct, an afferent arteriole, an efferent arteriole, or a renal granular cell) thereby modulating one or more renal functions. It is further believed that the modulation of the one or more renal functions, in turn, results in nondisease-indicative levels and/or reduced disease-indicative levels of the one or more renal function-associated parameters.

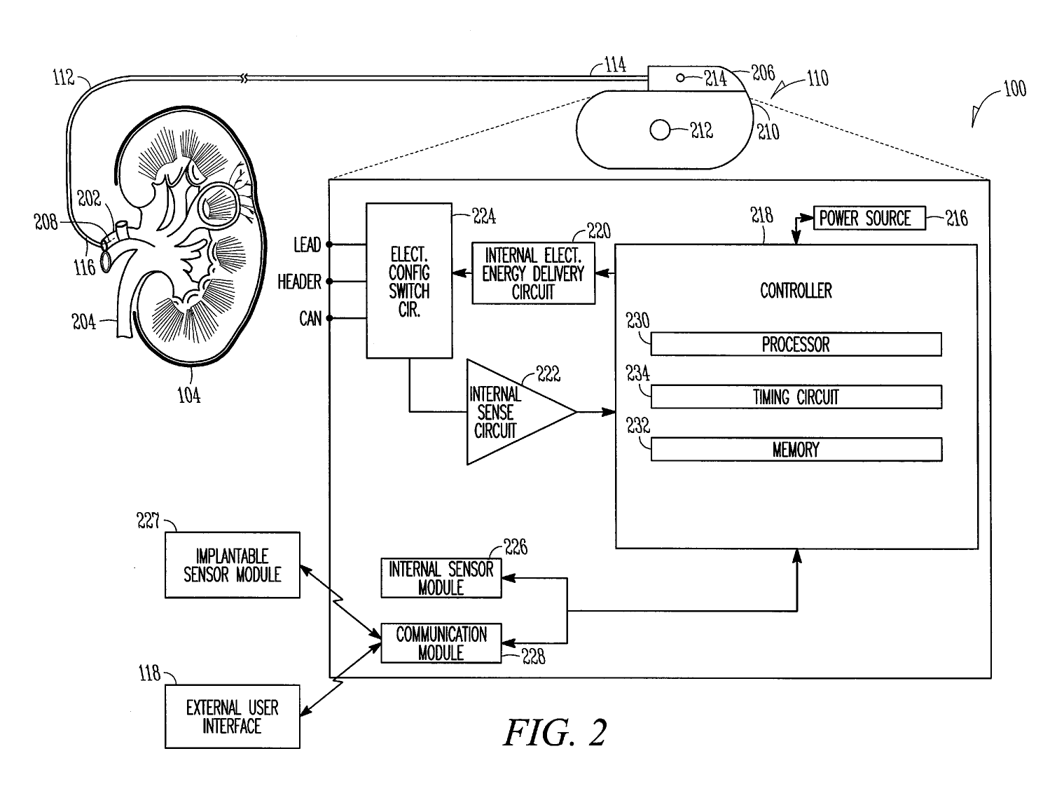

The simplified block diagram of FIG. 2 illustrates one conceptual embodiment of the system 100, which may deliver the electrical energy stimulation to the subject's 102 (FIG. 1) kidney(s) 104. As shown, the system 100 includes an IMD 110, such as a pulse generator, coupled via one or more leads 112 to a kidney 104, such as the left kidney 104. In this embodiment, the one or more leads 112 are providing vascular access to the kidney 104 via a renal vein 202. In another embodiment, the one or more leads 112 may be provided access to the kidney 104 via a ureter 204 access.

Each lead 112 extends from a lead proximal end portion 114, which is coupled to an insulating header 206 of the IMD 110, to a lead distal end portion 116, positioned within the renal region. Each lead distal end portion 116 includes one or more electrodes 208 for delivering the electrical energy stimulation generated by the IMD 110. The one or more electrodes 208 may also be used for sensing information about one or more renal function-associated parameters, which may then be used by the IMD 110 (e.g., a processor 230) to calculate a kidney status indicative signal, which indicates at least one of the absence, presence, increase, decrease, occurrence, termination, impending change, or rate of change of one or more renal functions. The kidney status indicative signal may in turn be used for proper electrical energy stimulation generation and delivery (e.g., an energy injection location, an energy injection duration, an energy injection intensity, and energy injection frequency, an energy injection polarity, an energy injection electrode configuration, or an energy injection waveform). In addition to the lead electrodes 208, other electrodes usable in the delivery of the electrical energy stimulation may be located on a hermetically-sealed enclosure 210 of the IMD 110 (typically referred to as a can electrode 212) or on the insulating header 206 (typically referred to as a header electrode 214).

As shown, the IMD 110 includes electronic circuitry components that are enclosed within the hermetically-sealed enclosure 210, such as a controller 218, a power source 216, an electrical energy delivery circuit 220, an internal sense circuit 222, an electronic configuration switch circuit 224, an internal sensor module 226, and a communication module 228. The power source 216 provides operating power to all of the aforementioned IMD internal modules and circuits. In certain embodiments, the power source 216 should be capable of operating at low current drains for long periods of times.

The controller 218 includes, among other things, a processor 230, a memory 232, and a timing circuit 234. The processor 230 is configured to determine an electrical energy signal command using information about a desired biological response of one or more renal function-associated parameters. The electrical energy signal command is subsequently communicated to the electrical energy delivery circuit 220, which is configured to generate an electrical energy signal deliverable by one or more chosen electrodes 208, 212, or 214 to the kidney 104. In various examples, the one or more delivery electrodes are chosen such that a substantially large portion of the electrical energy signal passes through one or more kidney structures (e.g., a glomerulus, a Bowman's capsule, a macula densa, a tubule, a peritubular capillary network, a collecting duct, an afferent arteriole, an efferent arteriole, or a renal granular cell). The electrical energy circuit 220 is selectively coupled to the one or more electrodes 208, 212, or 214 by the electronic configuration switch circuit 224.

The electrical energy stimulation may be delivered to the kidney 104 in various ways. For instance, the electrical energy stimulation delivered to the kidney 104 by the electrodes 208, 212, or 214 includes a frequency equal to or greater than about 1 KHz. In one such embodiment, the signal frequency equal to or greater than about 1 KHz is delivered in one or more bursts having a burst frequency substantially less than 1 KHz, such as around 1 Hz. In another embodiment, the electrical energy stimulation delivered to the kidney 104 by the electrodes 208, 212, or 214 includes a frequency of greater than about 50 KHz. In yet another embodiment, the electrical energy stimulation delivered to the kidney 104 by the electrodes 208, 212, or 214 includes a continuous periodic or pulsed periodic electric current or voltage. In still other embodiments, the electrical energy stimulation may include a frequency substantially below 1 KHz.

The internal sense circuit 222 and the internal sensor module 226 (i.e., one or more measurement units) are configured to sense information about then-current values of the one or more renal function-associated parameters. From the internal sense circuit 222 and the internal sensor module 226, the parameter information is sent to the controller 218 for processing (e.g., calculation of a kidney status indicative signal) by the processor 230. The processor 230 may compare the then-current values of the one or more renal function-associated parameters (or then-current kidney indicative signal) to the desired parameter values (or desired kidney indicative signal) stored in the memory 232 and thereafter determine whether the electrical energy stimulation command communicated to the energy delivery circuit 220 needs to be adjusted or terminated.

The system 100 of this embodiment further includes an external user-interface 118 and an implantable sensor module 227 (i.e., measurement units or display devices not physically connected to the IMD 110). The external user-interface 118 receives, for example, manually entered desired values of the one or more renal function-associated parameters and communicates the same to the IMD 110 via the communication module 228. The manually entered values may be used in lieu of preprogrammed parameter values stored in the memory 232. The implantable sensor module 227 includes sensors to measure information about then-current values of the one or more parameters and relays such information to the IMD 110 via the communication module 228.

It is to be noted that FIG. 2 illustrates just one conceptualization of various modules, circuits, and interfaces of system 100, which are implemented either in hardware or as one or more sequences of steps carried out on a microprocessor or other controller. Such modules, circuits, and interfaces are illustrated separately for conceptual clarity; however, it is to be understood that the various modules, circuits, and interfaces of FIG. 2 need not be separately embodied, but may be combined or otherwise implemented.

FIG. 3A schematically illustrates the system 100 in the process of delivering electrical energy stimulation in the form of an electric current 304 and an associated electric field 306 to the subject's kidney 104. In certain embodiments, the electrical energy stimulation includes a pulsed voltage signal with approximately a zero average amplitude, a frequency between approximately 1 KHz and approximately 1 MHz, and a peak-to-peak amplitude sufficient to produce an electric field strength of approximately 10 volts per centimeter. As shown, the kidney 104 is a bean-shaped structure, the rounded outer convex of which faces the side of the subject's body 102 (FIG. 1). The inner, indented surface of the kidney 104, called the hilum, is penetrated by a renal artery, a renal vein 202, nerves, and a ureter 204, which carries urine out of the kidney 104 to the bladder (see FIG. 1). As shown, the system 100 includes an IMD 110 electrically coupled to the kidney 104 via at least one lead 112. The lead extends from a lead proximal end portion 114, where it is coupled to an insulated header 206 of the IMD 110, to a lead distal end portion 116 disposed within the renal vein 202. In this embodiment, the lead 112 is provided vascular access to the renal vein 202 via the inferior vena cave 302. In another embodiment, the lead distal end portion 116 is positioned deep within the kidney 104, such as in an arcuate vein, an interlobar vein, or a segmental vein. In yet another embodiment, the lead 112 may be delivered via a urethra-bladder-ureter 204 access.

As shown, but as may vary, the lead distal end portion 116 includes at least one implanted electrode 208 disposed proximal to the kidney 104 (i.e., within, on, or about the kidney 104), while the hermetically-sealed enclosure 210 (via can electrode 212) or the insulating header 206 (via header electrode 214) acts as another implanted electrode by being at least partially conductive. In this way, an electrical energy signal provided by the IMD 110 and delivered by the lead electrode 208 disposed within, on, or about the kidney 104 may return through a portion of the kidney to the can 212 or header 214 electrode. In certain embodiments, the electrical energy stimulation is delivered in the form of an electric current 304 having an associated electric field 306.

The electric current 304 and the associated electric field 306 may be positioned such that one or more structures of the kidney 104 (e.g., a glomerulus, a Bowman's capsule, a macula densa, a tubule, a peritubular capillary network, a collecting duct, an afferent arteriole, an efferent arteriole, or a renal granular cell) are immersed within the current 304 or field 306 sufficient to affect one or more renal functions, and more specifically, affect one or more parameters associated with the one or more renal functions (e.g., an electrolyte level, a water level, a metabolic waste level (including a creatinine level, a blood urea nitrogen level, or a uric acid level), a pharmacological agent level, a hormone level, a blood pressure level, an erythropoietin level, a vitamin D level, a glucose level, a pH level, or a glomerulus filtration rate level). The present system 100 is adapted to work in a variety of electrode configurations and with a variety of electrical contacts (e.g., patches) or electrodes in addition to the electrode configuration shown in FIG. 3A. For instance, multiple leads 112 may be placed in different kidney locations to improve the electric current 304 or electric field 306 distributions. Alternatively or additionally, lead 112 may have one or more additional electrodes wherein the one or more electrodes perform as the cathode for the electric current 304 and associated electric field 306, for example.

FIG. 3B diagrammatically illustrates one of many nephrons 350 in a kidney 104 (FIG. 1). As discussed above, the nephrons 350 perform the actual filtering in the kidneys 104. It follows that in order to modulate one or more functions of the kidney 104, the function of one or more nephrons 350 or their associated structures need to be modulated. For better understanding of how the present subject matter may be used to affect one or more renal functions, discussion will now turn to the modulation of a nephron 350 and its associated structure.

Each nephron consists of a spherical filtering component, called the renal corpuscle 352, and a tubule 354 extending from the renal corpuscle 352. The renal corpuscle 352 is responsible for the initial step in urine formation (i.e., the separation of a protein-free filtrate from plasma) and consists of interconnected capillary loops (the glomerulus 356) surrounded by a hollow capsule (Bowman's capsule 358). Blood enters and leaves Bowman's capsule 358 through afferent and efferent arterioles 360, 362 that penetrate the surface of the capsule 358. Proximal to the arterioles 360, 362 are one or more renal granular cells 361, the latter of which stimulate the release of renin upon change in systemic blood pressure. A fluid-filled space exists within the capsule 358, and it is into this space that fluid filters. Opposite the vascular pole, Bowman's capsule 358 has an opening that leads into the first portion of the tubule 354. Specialized cells in the thick ascending limb of the tubule 354 closest to the Bowman's capsule 358 constitute the macula densa 363, which generates signals that influence the rennin-angiotensin system. The filtration barrier in the renal corpuscle 352 through which all filtered substances pass consists of three layers: the capillary endothelium of the glomerular capillaries, a basement membrane, and a single-celled layer of epithelial cells.

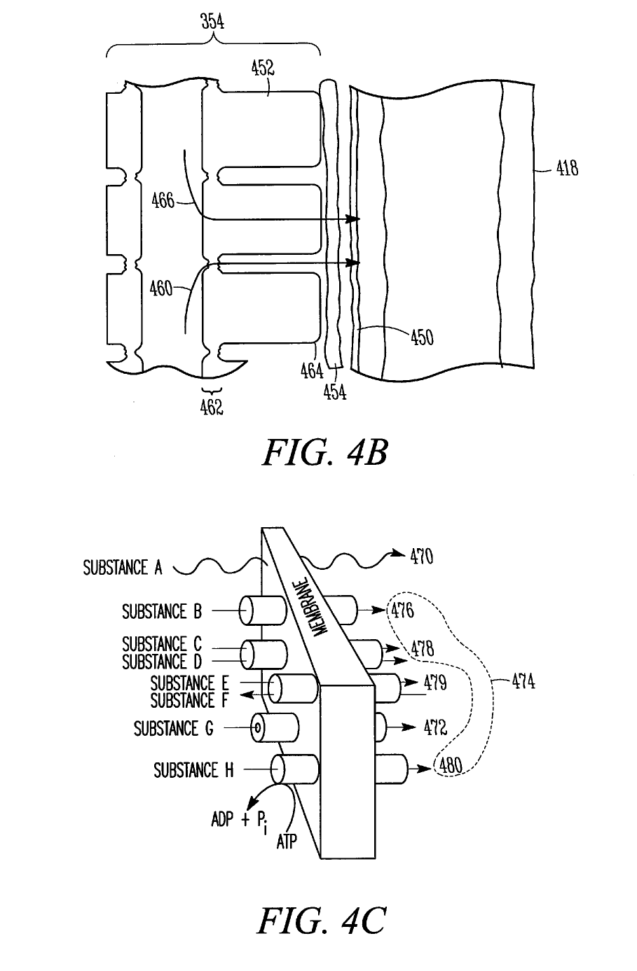

FIG. 4A illustrates portions of the renal process 400, which includes glomerular filtration 410, tubular secretion 412, tubular reabsorption 414, and excretion 416. Urine formation begins with glomerular filtration 410, which includes the bulk flow of fluid from the glomerular capillaries 402 into Bowman's capsule 358. Many low-molecular weight components of blood are freely filtered during glomerular filtration 410. Among the most common substances included in the freely filtered category are the ions sodium, potassium, chloride, and bicarbonate; the neutral organics glucose and urea; amino acids; and peptides like insulin and antidiuretic hormone (ADH).

As the filtrate flows from Bowman's capsule 358 through the various portions of the tubule 354, its composition is altered, mostly by removing material (tubular reabsorption 414) but also by adding material (tubular secretion 412). The tubule 354 is, at all points, intimately associated with peritubular capillaries 418, a relationship that permits the transfer of materials between the capillary plasma and the lumen of the tubule 354. As shown in FIG. 4B, the basic processes of tubular reabsorption 414 and tubular secretion 412 involve crossing two barriers: the tubular epithelium 452 and the endothelial cells 450 lining the peritubular capillaries 418.

For reabsorbed substances, the endothelial cell barrier 450 is like the barrier of many other peripheral capillary beds in the body—solutes cross the peritubular capillary barrier through the basement membrane 454 and then the fenestrae in the endothelial cells 450. For secreted substances, crossing the endothelium 450 is similar to the filtration process in the glomerular capillaries 402 (FIG. 4A), but it is traveling in the opposite direction. However, because the endothelium 450 is highly permeable to small solutes, this is quite feasible providing there is a suitable concentration gradient.

Crossing the epithelium 452 lining the tubule 354 can be performed in a single step or in two steps. The paracellular route 460 (single step) is when the substance goes around the cells (i.e., through the matrix of the tight junctions that link each epithelial cell 452 to its neighbor). More typically, however, the substances travel through the cells in a two-step process—across the apical membrane 462 facing the tubular lumen and across the basolateral membrane 464 facing the interstitium. This is called the transcellular route 466.

Arrays of mechanisms exist by which substances cross the various barriers. Renal cells use whichever set of tools is most suitable for the task. The general classes of mechanisms for traversing the barriers are illustrated in FIG. 4C and include movement by diffusion 470, movement through channels 472, and movement by transporters 474.

Diffusion 470 is the random movement of free molecules in a solution. Net diffusion 470 occurs across a barrier if there is a driving force, such as a concentration gradient, or for charged molecules, a potential gradient, and if the barrier is permeable. This applies to almost all substances crossing the endothelial barrier 450 (FIG. 4B) lining the peritubular capillaries 418 (FIG. 4B). It applies to substances taking the paracellular route 460 (FIG. 4B) around the tubular epithelium 452 (FIG. 4B) and to some substances taking the transcellular route 466 (FIG. 4B). Substances that are lipid solute, such as the blood gases or steroids, can diffuse directly through the lipid bilayer.

Most substances that are biologically important cannot penetrate lipid membranes. To cross a membrane, they need to move through specific integral membrane proteins, which are dividend into categories of channels 472 and transporters 474. Channels 472 are small pores that permit, depending on their structure, water or specific solutes to diffuse through them. Examples of specific channels 472 include sodium channels and potassium channels that permit diffusion of these molecular species. Movement through channels 472 is passive (i.e., no external energy is required). The energy to drive the diffusion is inherent in the concentration gradient or, more specifically, the electrochemical gradient, because ions are driven through channels and around cells via the paracellular route 460 not only by gradients of concentration but also by gradients of voltage. Channels 472 represent a mechanism for rapidly moving across membranes large amounts of substances, which would otherwise diffuse slowly or not at all. The amount of material passing through an ion channel 472 can be controlled by opening and closing the channel pore.

Transporters 474, like channels 472, permit the transmembrane flux of a solute that is otherwise impermeable in the lipid bilayer. However, unlike channels 472, many transporters 474 are extremely specific, transporting only 1 or at most a small class of substances. The specificity is usually coupled to a lower rate of transport because the transported solutes bind much more strongly to the transport protein. Furthermore, the protein must undergo a more elaborate cycle of conformational change to move the solute from one side of the membrane to the other.

Transporters 474 can be grouped into categories including uniporters 476, symporters 478 and antiporters 479, and primary active transporters (ATP) 480 according to basic functional properties. Uniporters 476 permit movement of a single solute species through the membrane. Movement through a uniporters 476 is like diffusion in that it is driven by concentration gradients, but is different in that the transported material moves through the uniporter protein rather than the membrane. Symporters 478 and antiporters 479 move two or more solute species in the same direction across a membrane (symporters) or in opposite directions across a membrane (antiporters). With symporters 478 and antiporters 479, at least one of the solutes moves down its electrochemical gradient and provides the energy to move one or more of the other solutes up its electrochemical gradient. Primary active transporters 480 are membrane proteins that are capable of moving one or more solutes up their electrochemical gradients, using the energy obtained from the hydrolysis of adenosine triphosphate (ATP). Among the key primary active transporters in the kidney 104 (FIG. 1) is Na—K-ATPase (often referred to as the “sodium pump”), some form of which is present in all cells of the body. This transporter simultaneously moves sodium against its electrochemical gradient out of a cell and potassium against its gradient into a cell.

In light of the above-discussed systems 100 (FIGS. 1, 2, 3A) and further in light of the above-discussed kidney structures, including the glomerulus 356, the Bowman's capsule 358, the tubule 354, the peritubular capillary network 418, the collecting duct, the afferent arteriole 360, the efferent arteriole 362, or the renal granular cell 361, some beliefs of how the electrical energy stimulation may be targeted toward renal function modulation, and thus renal solute control, renal water control, and renal system blood pressure (i.e., examples of renal function-associated parameters) are discussed below.

Electrical Energy Stimulation Targeted Toward Renal Solute Control:

Electrical modulation of glomerular filtrate solute control may be targeted toward the filtration by the glomerulus 356 (FIG. 3B). Alternatively or additionally, concentration of a particular solute may be modulated via imparting electrical energy stimulation across various channels 472 (FIG. 4C) (e.g., sodium channel, potassium channel) or transporters 474 (FIG. 4C) (e.g., uniporter 476, symporter 478 and antiporter 479) with the tubule 354 (FIG. 4A) or collecting duct.

As discussed above, movement through channels 472 is passive as the diffusion therethrough is due, in part, to specific solute concentration gradients, and more specifically, to the electrochemical gradient. Ions are driven through and around channels 472 not only by gradients due to the specific solute concentration, but also by gradients of voltage across the channel 472. This sensitivity of channels 472 to voltage provides a mechanism to support the belief that channels 472 may be modulated via applied electrical energy stimulation. Regarding transporters 474, it has been shown, such as in Blank, M. and Soo, L., Threshold for inhibition of Na, K-ATPase by ELF alternating currents, Bioelectromagnetics, Vol. 13, Issue 4 (Published Online October 2005): 329-333, that alternating current can increase or decrease the ATP-splitting activity of the membrane enzyme Na—K-ATPase.

As further discussed above, maintenance of proper blood electrolyte (e.g., sodium, chlorine, or potassium) levels is a key function of the kidney. Supporting the premise that modulation of ion channels within the nephrons is possible includes studies, such as Teissie, J. and Tsong, T., Voltage Modulation of Na+/K+ Transport in Human Erythrocytes, Journal of Physiology (Paris), (May 1981); 77(9): 1043-1053 PMID: 6286955; Serpersu, E. H. and Tsong, T. Y., Activation of electrogenic Rb+ transport of (Na K)-ATPase by an electric field, J. Biol. Chem., (Jun. 10, 1984); 259(11): 7155-62; Liu, D. S., Astumian, R. D., and Tsong, T. Y., Activation of Na+ and K+ pumping modes of (Na, K)-ATPase by an oscillating electric field, J. Biol. Chem., (May 5, 1990); 265(13): 7260-7 PMID: 2158997; and Serpersu, E. H., Tsong, T. Y., Stimulation of a ouabain-sensitive Rb+ uptake in human erthrocytes with an external electric field, J. Membr. Biol., (1983); 74(3): 191-201 PMID: 6887232, noting that modulation of sodium and potassium ion channels in human erythrocytes (red blood cells) via electrical energy stimulation has been accomplished. Further, ion channels in human erythrocytes can be selectively targeted by altering the frequency of the applied electrical energy stimulation. Specifically, sodium has been found to be sensitive to frequencies from 1 KHz to 100 KHz and potassium channels have been found to be sensitive to frequencies of about 1 MHz. (See Serpersu, E. H. and Tsong, T. Y., Activation of electrogenic Rb+ transport of (Na K)-ATPase by an electric field and Liu, D. S., Astumian, R. D., and Tsong, T. Y., Activation of Na+ and K+ pumping modes of (Na, K)-ATPase by an oscillating electric field, J. Biol. Chem. The electric fields required to produce these effects are on the order of 10 V/cm. The half life of an ion channel opening due to applied electric fields is about 10 seconds (see Serpersu, E. H. and Tsong, T. Y., Activation of electrogenic Rb+ transport of (Na K)-ATPase by an electric field); thus, continuous application of electrical energy stimulation may not be required.

In addition to transcellular 466 (FIG. 4B) routes, solute movement may also occur via paracellular 460 (FIG. 4B) routes. It has been found that both solute concentrations and electric fields 306 (FIG. 3A) play a role in paracellular solute movement. Solutes that can move via paracellular routes include urea, potassium, chloride, calcium, and magnesium. Paracellular 460 route sensitivity to electric fields 306 may allow modulation of these routes via imposition of electrical energy stimulation.

In addition to the work noted above with human erythrocytes, work, such as Burkhoff, D., Shemer, I., Felzen, B., Shimizu, J., Mika, Y., Dickstein, M., Prutchi, D., Darvish, N., Ben-Haim, S. A., Electric currents applied during the refractory period can modulate cardiac contractility in vitro and in vivo, Heart Failure Rev., (January 2001); 6(1): 27-34 PMID: 11248765, has been conducted with cardiac contractility modulation via application of electric current during cardiac refractory periods. Application of electric current has been shown to modify calcium movement across cellular membranes during certain phases of cardiac myocyte action potential.

Glomerular filtration is dependent on solute size, hydrostatic and oncotic pressures and the electrical charge of individual solutes. For any given size, negatively charged macromolecules are filtered to a lesser extent, and positively charged macromolecules to a greater extent, then neutral molecules. The filtrate dependence on the solute's electrical charge is due to fixed negative charge within certain portions of the glomerular membrane. It is important to note that charge dependent filtration pertains only to macromolecules (e.g., albumin) and not mineral ions or low weight molecules (e.g., chloride or bicarbonate ions). It has been shown, such as in Kverneland, A., Feldt-Rasmussen, B., Vidal, P., Welinder, B., Bent-Hansen, L., Soegaard, U., and Decker, T., Evidence of changes in renal charge selectivity in patients with type 1 (insulin-dependent) diabetes mellitus, Diabetologia, (September 1986): (9) 634-9, that alterations in the glomerular membrane charge influences filtration of albumin resulting in albuminuria. Thus, it may be possible to alter glomerular filtration of certain charged macromolecules by imposing electric fields 306 across the glomerulus 356.

Electrical Energy Stimulation Targeted Toward Renal Water Control:

Approximately 99% of the water in the glomerular filtrate is reabsorbed by the kidneys 104 (FIG. 1). Reduction of the reabsorbed 414 (FIG. 4A) portion of water within the nephrons 350 (FIG. 3B) via application of electrical energy stimulation provides an opportunity to promote diuresis. Like conventional pharmaceutical diuretics, diuresis via imposition of electrical energy stimulation could be caused by increased excretion of sodium, which as noted above, may be manipulated via application of electrical energy stimulation.

Another potential method to promote diuresis is the application of electrical energy stimulation in a manner that modulates the peritubular capillary's 418 (FIG. 4A) aquaporin sensitivity to antidiuretic hormone (ADH). Reducing the kidneys' 104 sensitivity to ADH will promote diuresis. ADH is secreted by the posterior pituitary and acts on the peritubular capillary of the kidneys 104 to cause them to reabsorb water, thereby concentrating the urine. Since it is believed that most aquaporins are virtually impermeable to ions, control of aquaporin function via application of electrical energy stimulation may be difficult. If aquaporin function is insensitive to applied electrical energy stimulation, it would be advantageous in one regard since it prevents unintentional change in aquaporin function when the electrical energy stimulation is targeted at other renal structures.

Electrical Energy Stimulation Targeted Toward Systemic Blood Pressure:

Renal control of blood pressure results from both the regulation of blood volume within the vascular tree via control of sodium and water (e.g., using the techniques discussed above) and by the excretion of chemical agents, such as rein and angiotension II, that alter vascular resistances to correct blood pressure. Renin, for example, is released by renal granular cells 361 (FIG. 3B). It is believed that the release of renin by the renal granular cells 361 may be accomplished via application of electrical energy stimulation.

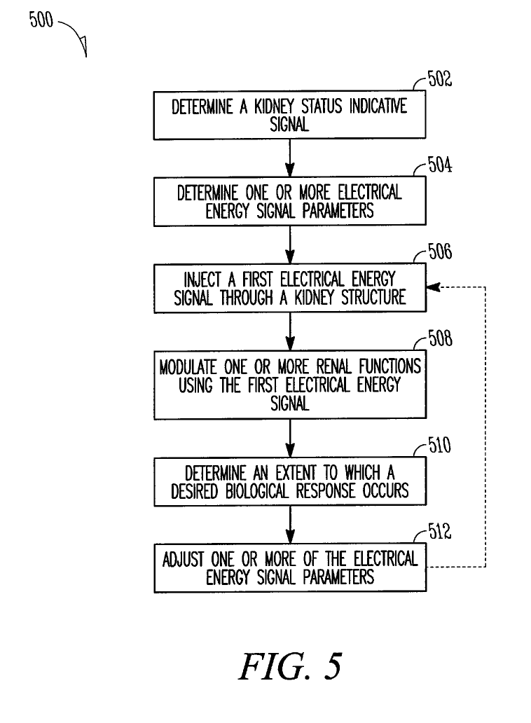

FIG. 5 illustrates a method 500 of modulating one or more renal functions by applying electrical energy stimulation to one or more kidney structures (e.g., a glomerulus, a Bowman's capsule, a macula densa, a tubule, a peritubular capillary network, a collecting duct, an afferent arteriole, an efferent arteriole, or a renal granular cell). At 502, a kidney status indicative signal is determined. Determination may be from, for example, an internal sensor module 226 (FIG. 2), an implantable sensor 227 (FIG. 2) or information communicated to the IMD 110 (FIG. 2) via an external user interface 118 (FIG. 2). In certain embodiments, the kidney status indicative signal includes information about one or more renal function-associated parameters, such as whether a then-current value of the one or more parameters is associated with a current or impending disease state. If it is determined that one or more renal function-associated parameters values are indicative of disease, one or more electrical energy signal parameters aimed at normalizing the parameters are determined at 504. In various embodiments, the one or more electrical energy signal parameters include an energy injection location, an energy injection duration, an energy injection intensity, an energy injection frequency, an energy injection polarity, an energy injection electrode configuration, or an energy injection waveform. In certain embodiments, the electrical energy signal includes a pulsed voltage signal with approximately a zero average amplitude, a frequency between approximately 1 KHz and approximately 1 MHz, and a peak-to-peak amplitude sufficient to produce an electric field strength of approximately 10 volts per centimeter.

At 506, a first electrical energy signal characterized by the one or more electrical energy stimulation parameters is internally injected between a first and a second electrode, such that a substantially large portion of the signal flows through a subject's kidney(s), and more specifically, at least one of the glomerulus, the Bowman's capsule, the macula densa, the tubule, the peritubular capillary network, the collecting duct, the afferent arteriole, the efferent arteriole, or the renal granular cell. At 508, one or more renal functions are modulated using the first electrical energy signal. In various embodiments, modulation of the one or more renal functions includes affecting a change of the one or more renal function-associated parameters (e.g., an electrolyte level, a water level, a metabolic waste level, a pharmacological agent level, a hormone level, a blood pressure level, an erythropoietin level, a vitamin D level, a glucose level, a pH level, or a glomerulus filtration rate level).

At 510, an extent to which the desired biological response of the one or more renal function-associated parameters occurs is determined. In certain embodiments, this may include a re-determination of the kidney status indicative signal and comparison of such signal with stored desired parameter values. At 512, one or more of the electrical energy signal parameters may optionally be adjusted in light of the extent determined at 510. The process may subsequently return to 506 for further electrical energy stimulation.

Renal function modulation via application of electrical energy stimulation is discussed herein. The electrical energy stimulation may be used to supplement or in lieu of existing renal failure treatments (e.g., drug therapy, hemodialysis, or transplantation) to keep kidney subjects in a state of relative well-being by preventing, delaying, or minimizing renal conditions including, for example, chronic kidney disease and end stage renal failure. It is believed that by selectively manipulating one or more kidney structures that one or more renal functions may be modulated in a desired way, such as the way non-disease state kidneys would normally function. By modulating the one or more renal functions, a desired biological response of one or more renal function-associated parameters may be effectuated, thereby treating or preventing associated diseases (e.g., hypertension, edema, heart failure, blood electrolyte imbalances, and others).

It is to be understood that the above description is intended to be illustrative, and not restrictive. For instance, while a majority of the foregoing discusses electrical energy stimulation in the form of an electric current or an associated electric field, the present subject matter may also include other forms of electrical energy stimulation, such as magnetic fields or magnetic flux to modulate one or more renal functions. For instance, according to at least one study, such as is found in Blank, M. and Soo L., Frequency Dependence of NA, K-ATPase Function in Magnetic Fields, Bioelectrochemistry and Bioenergetics, May 1997: 42(2) 231-234, Na—K-ATPase function has been found to be dependent on magnetic energy.

Although specific embodiments have been illustrated and described herein, it will be appreciated by those of ordinary skill in the art that any arrangement which is calculated to achieve the same purpose may be substituted for the specific embodiment shown. This patent document is intended to cover adaptations or variations of the present subject matter. It is to be understood that the above description is intended to be illustrative, and not restrictive. Combinations of the above embodiments and other embodiments will be apparent to those of skill in the art upon reviewing the above description. The scope of the present subject matter should be determined with reference to the appended claims, along with the full scope of equivalents to which such claims are entitled.

What is claimed is:

1. A method for applying a stimulus to at least one of a glomerulus, a Bowman's capsule, a macula densa, a tubule, a peritubular capillary network, a collecting duct, an afferent arteriole, an efferent arteriole, or a renal granular cell within a kidney of a subject, the method comprising:
injecting a first electrical energy signal having a frequency equal to or greater than about 1 KHz between a first electrode and a second electrode, including passing a substantially large portion of the first electrical energy signal through at least one of the glomerulus, the Bowman's capsule, the macula densa, the tubule, the peritubular capillary network, the collecting duct, the afferent arteriole, the efferent arteriole, or the renal granular cell;
modulating one or more renal functions using the first electrical energy signal; and
wherein at least one of the first electrode or the second electrode is disposed within the subject and proximal to the kidney.

2. The method of claim 1, wherein injecting the first electrical energy signal includes injecting the signal frequency equal to or greater than about 1 KHz in one or more bursts having a burst frequency less than 1 KHz.

3. The method of claim 1, wherein injecting the first electrical energy signal includes injecting a signal frequency greater than about 50 KHz between the first electrode and the second electrode.

4. The method of claim 1, further comprising measuring one or more parameters associated with the one or more renal functions.

5. The method of claim 4, wherein measuring the one or more parameters associated with the one or more renal functions include measuring one or more of an electrolyte level, a water level, a metabolic waste level, a pharmacological agent level, a hormone level, a blood pressure level, an erythropoietin level, a vitamin D level, a glucose level, a pH level, or a glomerulus filtration rate level.

6. The method of claim 4, further comprising determining a kidney status indicative signal using information about the one or more parameters associated with the one or more renal functions; and
wherein the kidney status indicative signal indicates at least one of the absence, presence, increase, decrease, occurrence, termination, impending change, or rate of change of the one or more renal functions.

7. The method of claim 4, further comprising controlling the first electrical energy signal using information about the one or more parameters associated with the one or more renal functions.

8. The method of claim 7, wherein controlling the first electrical energy signal includes determining one or more of an energy injection location, an energy injection duration, an energy injection intensity, an energy injection frequency, an energy injection polarity, an energy injection electrode configuration, or an energy injection waveform of the first electrical energy signal using information about the one or more parameters associated with the one or more renal functions.

9. The method of claim 7, wherein controlling the first electrical energy signal includes determining an extent to which a desired response of the one or more parameters associated with the one or more renal functions occurs.

10. The method of claim 9, further comprising adjusting one or more of an energy injection location, an energy injection duration, an energy injection intensity, an energy injection frequency, an energy injection polarity, an energy injection electrode configuration, or an energy injection waveform of the first electrical energy signal using the determined extent to which the desired response of the one or more parameters occurs.

11. The method of claim 1, further comprising injecting a second electrical energy signal through at least a portion of a pulmonary region, a cardiac region, or a brain region.

12. The method of claim 1, wherein injecting the first electrical energy signal includes applying a voltage to the first electrode and the second electrode.

13. The method of claim 1, wherein injecting the first electrical energy signal includes injecting an electric current between the first electrode and the second electrode.

14. A system for applying a stimulus to at least one of a glomerulus, a Bowman's capsule, a macula densa, a tubule, a peritubular capillary network, a collecting duct, an afferent arteriole, an efferent arteriole, or a renal granular cell within a kidney of a subject, the system comprising:
a first electrode and a second electrode, at least one of the first electrode or the second electrode being configured for disposition within the subject and proximal to the kidney;
an electrical energy delivery circuit coupled to the first electrode and the second electrode, the electrical energy delivery circuit configured to generate a first electrical energy signal having a frequency between about 1 KHz and about 1 MHz;
wherein the first electrode and the second electrode are configured to direct a substantially large portion of the first electrical energy signal through at least one of the glomerulus, the Bowman's capsule, the macula densa, the tubule, the peritubular capillary network, the collecting duct, the afferent arteriole, the efferent arteriole, or the renal granular cell; and
wherein the first electrical energy signal having the frequency between about 1 KHz and about 1 MHz is configured to modulate one or more renal functions.

15. The system of claim 14, further comprising a measurement unit configured to measure one or more parameters associated with the one or more renal functions.

16. The system of claim 15, wherein the one or more measured parameters associated with the one or more renal functions include one or more of an electrolyte level, a water level, a metabolic waste level, a pharmacological agent level, a hormone level, a blood pressure level, an erythropoietin level, a vitamin D level, a glucose level, a pH level, or a glomerulus filtration rate level.

17. The system of claim 15, further comprising a processor coupled with the electrical energy delivery circuit, the processor configured to control the electrical energy delivery circuit using information about the one or more parameters associated with the one or more renal functions.

18. The system of claim 17, wherein the control of the electrical energy delivery circuit includes control of one or more of an energy injection location, an energy injection duration, an energy injection intensity, an energy injection frequency, an energy injection polarity, an energy injection electrode configuration, or an energy injection waveform of the first electrical energy signal using information about the one or more parameters associated with the one or more renal functions.

19. The system of claim 15, further comprising an external user interface unit communicatively coupled to the processor, the external user interface unit configured to at least one of display information about the one or more parameters associated with the one or more renal functions, provide an input of the subject's health related information, or allow external control of the electrical energy signal.

20. The system of claim 14, wherein at least one of the first electrode or the second electrode are disposed on a renal vasculature insertable lead.

21. The system of claim 14, wherein at least one of the first electrode or the second electrode are disposed on a urethra insertable lead.

22. The system of claim 14, wherein the electrical energy delivery circuit is disposed, at least in part, within an implantable medical device.

23. The system of claim 22, wherein at least one of the first electrode or the second electrode is disposed on a portion of the implantable medical device.

24. The system of claim 22, wherein the implantable medical device includes a cardiac therapy unit, the cardiac therapy unit configured to deliver at least one of a bradycardia therapy, a tachycardia therapy, or a cardiac resynchronization therapy to the subject.

25. The system of claim 14, wherein the first electrical energy signal includes a pulsed voltage signal having approximately a zero average amplitude and a peak-to-peak amplitude sufficient to produce an electrical field strength of approximately 10 volts per centimeter.
